(19) United States
(12) Patent Application Publication
(10) Pub. No.: US 2025/0050327 A1
(43) Pub. Date: Feb. 13, 2025

(54) PROCESSING DEVICE FOR PROCESSING A LIQUID SAMPLE

(71) Applicant: CYTENA BIOPROCESS SOLUTIONS CO., LTD, Taipei (TW)

(72) Inventors: Cheng-Han TSAI, Taipei (TW); Da-Han KUAN, Taipei (TW)

(21) Appl. No.: 18/718,572

(22) PCT Filed: Dec. 3, 2022
(86) PCT No.: PCT/EP2022/084321

§ 371 Date: Jun. 11, 2024

(30) Foreign Application Priority Data
Dec. 17, 2021 (LU) LU501039

(51) Int. Cl. B01L 3/00

(52) U.S. Cl. CPC B01L 3/5025; B01L 2200/026; B01L 2300/041; B01L 2300/0829; B01L 2400/0633

(57) ABSTRACT

The invention relates to a processing device (1) for processing of liquid sample contained in a multiwell carrier (2), wherein the processing device comprises a housing (3) with a bottom part (4) and a lid part (5), wherein the bottom part and the lid part delimit an inner space of the housing, and a connection part (6) for fluidically connecting the processing device to a processing unit (7) wherein the connection part is fluidically connected to the inner space, wherein the housing has several hollow extension tubes (9a, 9b), which extend from the bottom part and which are fluidically connected to the inner space. The processing device comprises at least one valve device (10a, 10b) that is at least partly arranged in the inner space of the housing and is moveable between an open position in which the connection part is fluidically connected with an outlet opening of an extension tube and a closed position in which the valve device fluidically disconnects the connection part from the outlet opening of the extension tube.

The invention relates to a processing device for processing a liquid sample. Additionally, the invention relates to a processing system comprising such a processing device and the use of the processing device or processing system in an incubator for biological sample.

A cell line development process is required for most biopharmaceutical development process. Cell line development is a method for selecting a cell line that produces protein/antibodies with high yield and optimized quality. However, the cell line development is a very complicated, labour-intensive, and costly process. Development time for most pharmaceutical products is half a year.

A core tool required in the cell line development is a bioreactor for cell incubation, which is also often indicated as incubator. Starting from incubating single cells, the scale of the cell culture is continually expanded until a high-volume production scale is reached through repeated transferring steps of selection and up-scaling. Therefore, for different phases and different quantities of cells, bioreactors of different sizes ranging from a bioreactor at a microliter level to a large-scale bioreactor at a production level are required. However, there is a limited ability to optimize the cell incubating environment of a smaller-sized bioreactor. An ideal bioreactor generally needs to enable cells to continually grow in suspension, dynamically monitor biosignals, and perform feedback control on dissolved oxygen content and pH value. It is difficult to achieve the foregoing functions in a microliter-leveled bioreactor.

A further core tool required in cell line development is a multiwell carrier that comprises a plurality of wells. Said multiwell carrier can be arranged in the aforementioned bioreactor. Each of the wells is used to contain a liquid sample that can comprise the cell or cells. Multiwell carriers comprising different number of wells are known (e.g. multiwell carriers having 24, 38, 96 or more wells). Additionally, processing devices are known that are attached to the multiwell carrier in order to process liquid sample. However, with the currently known processing devices it is only possible to process all wells at the same time. Thus, a well specific liquid sample processing is not possible. However, it is desirable that liquid samples being arranged in different wells are processed in a different manner. This is for example necessary if the liquid sample being arranged in different wells differ from each other.

The objective of the invention is to provide a processing device be means of which a well specific liquid processing is possible.

This objective is fulfilled by a processing device for processing of liquid sample contained in a multiwell carrier, wherein the processing device comprises
a housing with a bottom part and a lid part, wherein the bottom part and the lid part delimit an inner space of the housing, and a connection part for fluidically connecting the processing device with a processing unit wherein the connection part is fluidically connected with the inner space, wherein
the housing has several hollow extension tubes, which extend from the bottom part and which are fluidically connected with the inner space, characterized in that
the processing device comprises at least one valve device that is at least partly arranged in the inner space of the housing and is moveable between an open position in which the connection part is fluidically connected with an outlet opening of an extension tube and a closed position in which the valve device fluidically disconnects the connection part and the outlet opening of the extension tube.

The processing device according to the invention has the advantage that a well specific liquid processing is possible. This is possible due to the presence of the valve device. In particular, the position of the valve device sets whether the liquid sample can be processed or not. When the valve device is in the open position, the well into which the extension tube protrudes can be processed. However, when the valve device is the closed position, said well cannot be processed as the valve disconnects the fluid connection between the outlet opening of the extension tube and the connection part and, thus, the fluid connection between the liquid sample being in the well and the connection part.

The liquid sample depends on the usage field of the processing device. If the processing device is used in a bioreactor, the liquid sample comprises a biological particle. The biological particles may be microorganisms such as bacteria, archaean, yeast, fungi, and viruses or cells, DNA, RNA, or proteins. The liquid sample may comprise a single or multiple of the aforementioned biological particles. The liquid sample can contain a liquid and at least one biological particle. The liquid can promote the growth or the function of the biological particles arranged in the liquid. If the processing device is used in a chemical reactor, the liquid sample can comprise one or more chemical reagents.

A well is a receptacle for receiving the liquid sample. The well is part of a multiwell carrier. An outlet opening of the extension tube is the opening through which fluid leaves the processing device or enters the processing device.

Processing of liquid sample can comprise the supply or removal of fluid, in particular gas, into the well and/or mixing of the liquid sample being in the well and/or moving of the liquid sample and/or agitation of biological particles in the liquid sample. Moving of the liquid sample can be performed in order to apply shear stress to e.g. cells by means of the moved liquid. Mixing of the liquid sample is understood as a process in which the components of the liquid sample, in particular the biological particles, are moved relative to each other in such a way that a new arrangement pattern is created. The liquid processing can additionally or alternatively comprise a filtration of the liquid sample by means of a filter of the processing device.

The processing unit can control the pressure in the inner space. The processing unit can alternately apply positive or negative pressure on the liquid sample in order to process the liquid sample arranged in the well. Applying positive or negative pressure on the liquid sample can be performed successively multiple times.

According to an embodiment the valve device can be partially arranged in a cavity of the extension tube. The cavity fluidically connects the inner space of the processing device with the well into which the extension tube protrudes. The valve device can close an inlet opening of the extension tube when the valve device is arranged in the closed position. The inlet opening is the opening of the extension tube by means of which fluid, in particular gas, enters the cavity of the extension tube from the inner space. The inlet opening and the outlet opening are arranged at opposite ends of the extension tube.

All extension tubes of the processing device can be designed in the same way. Alternatively, the extension tubes can have a different diameter and/or shape. All extension tubes can be fixed to the bottom part. That means, the extension tube and the bottom part cannot move relative to each other. In particular, the extension tube can be integrated with the bottom part.

The valve device can move translatory from the open position to the closed position or vice versa. In particular, the valve device moves along a length axis of the extension tube. Thus, the valve device can be easily arranged within the existing processing device without redesigning the processing device. The valve device can move relative to the extension tube and/or the lid part and/or the bottom part between the open and close position. Additionally, the valve device can move from the open position to the closed position by means of gravity force. Thus, it is not necessary to provide any further means for bringing the valve device back to the closed position which simplifies the structure of the processing device. Additionally or alternatively forces, in particular mechanical forces, can be applied to the valve device to move the valve device from the open position to the closed position. The mechanical force can be applied to the valve device, e.g. by means of a spring to the valve device.

The valve device can have a sealing element for sealing an inlet opening of the extension tube when the valve device is arranged in the closed position. The sealing element can be circular and can surround the inlet opening of the extension tube when the valve device is arranged in the closed position. Thus, it can easily disconnect the fluid flow between the outlet opening of the extension tube and the connection part.

The valve device can have a valve head and a valve body. The sealing element can be arranged on or attached to the valve head. The valve head can be configured such that the valve head cannot enter the cavity of the extension tube. This means, the cross section of the valve head in a plane perpendicular to the length axis of the extension tube is greater than the cross section of the cavity so that the valve head cannot enter the cavity. The valve body is at least partially arranged in the cavity of the extension tube.

At least one guiding portion can be arranged at the valve body. The guiding portion and, thus, the valve body can be guided by the tube wall of the extension tube. In particular, the valve device can comprise several guiding portions that are arranged at the valve body. The guiding portions can be arranged at a distance to each other in circumferential direction of the valve body. The guiding portion can extend along a length axis of the valve device and/or protrude in radial direction from the valve body. In particular, the guiding portion can extend from a portion of the valve device comprising the sealing element to an end of the valve body distant to the sealing element. By provision of the guiding portion, in particular a plurality of guiding portions, the risk of jamming of the valve device is reduced. In particular, the extension tube can guide the movement of the valve device when it is moved from the open position to the closed position or vice versa. The valve device is configured such that at least a part of the valve body is arranged in the extension tube independent of whether the valve device is arranged in the closed or open position. Thus, the guidance of the valve device is ensured. All guiding portions can be identically designed.

The valve body can have a cylindrical portion. Additionally or alternatively the valve body can have a tapered portion. The tapered portion can be arranged closer to the outlet opening than the cylindrical portion. The provision of the tapered portion with its tip end arranged in the extension tube has the advantage that a condensed liquid of the liquid sample arranged in the well collects at the tip end and drips from the tip end back into the well due to gravity. A tapered tip end has the advantage that the volume and/or mass of the condensed liquid is reached faster than for example in a rectangular shape end so that the condensed liquid falls back sooner into the receptacle than in an embodiment in which the valve body has a rectangular shape.

The valve device can have a magnetic element. The magnetic element can be arranged at an end of the valve device distant to the extension tube. In particular, the magnet element can be connected with the valve head. In particular, the magnet element can be attached at the valve head. The magnet element can be a permanent magnet. By provision of the magnet element, the valve device can be moved from the closed position to the open position or vice versa as discussed below more in detail.

According to an embodiment the processing device can comprise a further connection part for, in particular fluidically, connecting the inner space with a measurement device. The measurement device can be a pressure sensor for measuring the pressure within the inner space. The measurement result can be used for controlling the processing system discussed below.

The connection part and/or the further connection part can protrude from the lid part and/or bottom part. Additionally, the connection part and/or the further connection part can protrude from the same housing side. Both connection parts can protrude in the same direction from the housing side.

The lid part can be fixed to the bottom part. “Fixed” means that the two parts are connected such that they cannot move relative to each other. The connection between the two said parts can be such that the connection between the lid part and the bottom part cannot be released without destroying the processing device, in particular the lid part and/or the bottom part. Due to said connection, the lid part and the bottom part cannot move relative to each other.

According to an embodiment the processing device is configured such that the lid part can comprise a lid frame and a membrane arranged on at least a part of the lid frame. A membrane is a component that has an extension in a x, y direction that is much greater than in the z-direction. The x, y, z-direction refers to Cartesian coordinate system with a x, y and z axis. The lid frame can be fixed to the bottom part. The membrane can be at least partially connected to the lid frame. In particular, the membrane can be bonded to the lid frame. This can be achieved in an easy manner if the outer rim of the membrane along the circumference of the membrane is bonded with the lid frame.

The lid frame can comprise a hole wherein a part of the valve device extends through the hole. The valve device, in particular the valve head, can be fixed to the lid part. In particular, the valve device, in particular the valve head, is, in particular directly, connected with the membrane. The valve device remains in connection with the lid part, in particular, independent of whether the valve device is arranged in the open or closed position. In said embodiment the valve device cannot move relative to the membrane. However, the valve device can move relative to the lid frame and/or the bottom part.

A part of the membrane can enter the hole when the valve device is arranged in the closed position. Thereto, the membrane has to be elastic. In particular, the lid frame can be made from a material that is stiffer than the membrane. Thus, an easy solution to how the valve device can be connected to the housing, in particular, the lid part, is provided. With this connection, jamming of the valve device and/or a movement of the valve device in an unwanted direction can be prevented in an easy way.

In an alternative embodiment the valve device is configured such that the valve device, in particular the valve head, is in contact with the lid part, in particular merely, when the valve device is arranged in the open position. In said embodiment the valve device is not fixed to the lid part. That means, in open position, the valve device is not connected to the lid part. In said embodiment the valve device is configured such that the valve device moves relative to the lid part and/or the bottom part. Such an embodiment has the advantage that no fixed connection has to be provided between the valve device and the lid part. Thus, the structure of the processing device is simpler as it is not necessary to provide an elastic membrane.

According to an embodiment the processing device can comprises an actuating element that is configured to act on the valve device so that the valve device moves from the closed position to the open position. The actuating element can be configured to exert force on the valve device while in the close position so that it moves into the open position. Likewise, the actuating element can be configured to exert force on the valve device so that the valve device moves from the open position to the close position. The actuating element can comprise a magnetic element. The magnetic element can be an electromagnet or a permanent magnet. Additionally, the magnetic element can be arranged at an end of the actuating element that faces the housing, in particular the lid part.

The actuating element can be moved relative to the processing device, in particular the lid part. Such an actuating element can comprise a permanent magnet. That means, the actuating element can be moved to an actuating position in which the magnet of the actuating element exerts force on the magnet of the valve device while in a close position. Said force leads to the valve device moving from the closed position to the open position. In order to move the valve device back to the closed position, the actuating element can be moved to a non-actuating position. In said position the magnet of the actuating element does not exert force on the magnetic element of the valve device so that the valve device moves back into the closed position.

In an embodiment in which the actuating element has an electromagnet, the actuating element can be configured such that it is arranged in the same position independent of whether the valve device is in the open position or the closed position. That means, the actuating element does not move relative to the housing, in particular the lid part. The electromagnet can be energized so that a force acts on the valve device resulting in a movement of the valve device from the closed position to the open position. For moving the valve device back to the closed position, the electromagnet is not energized or the electromagnet is energized such that a force acts on the valve device that supports the movement of the valve device back into the closed position.

The actuating element and the valve device can be arranged opposite to each other with respect to the lid part. This enables a simple structure of the processing device. In particular, it is possible that an actuating device of the processing device can be arranged on the processing device, in particular on the lid part. The actuating device can comprise an actuating housing wherein the actuating element is arranged at least partly in an inner space of the actuating housing. In particular, the magnetic element of the actuating element can be arranged outside the actuating housing. Additionally, the actuating element can move relative to the actuating housing.

The processing device can comprise several valve devices wherein each of the valve device is assigned to a hollow extension tube. With “assigned” it is meant that each of the valves is arranged such that it opens or closes a fluid connection between the connection part and the respective extension tube to which the valve is assigned. Each of the valves can be assigned to merely one extension tube. Thus, the number of valves can correspond with the number of extension tubes.

The valve devices can be configured to be moveable independently of each other. Thus, it is possible that the valve devices are arranged in different positions. This enables independent processing of the liquid samples arranged in different wells. The processing device can comprise several actuating elements to enable independent control of the valve devices. Each of the actuating elements can be assigned to a valve device. That means, each of the actuating elements can control the position of the valve device to which it is assigned. In particular, the actuating element can control the position of merely one valve device. The number of actuating elements can correspond with the number of valve devices. The actuating elements can be configured such that they actuate only on one valve device. That means, in embodiments which comprise several actuating elements and several valve devices it is secured such that the position of the valve device is controlled by merely the actuating element that is assigned to the valve device. Thus, a simple actuating device that controls the valve device positions is provided.

According to an aspect of the invention, a processing system is provided. The processing system comprises a processing device, in particular as described above, and a multiwell carrier, wherein the processing device is arranged such that at least one extension tube extends in a well containing the liquid sample of the multiwell carrier. The multiwell carrier comprises several wells. The processing system can be configured such that at least one extension tube extends into each well of the multiwell carrier, when the multiwell carrier comprises several wells.

The processing device can be, in particular directly, arranged on the multiwell carrier. Thus, a compact processing system can be achieved. The processing system can comprise the processing unit wherein the processing unit is configured to apply a positive pressure or a negative pressure in the inner space of the processing device. Additionally, the processing system can comprise a control unit for controlling the processing device and/or the processing unit. The control unit can be a processor or comprise at least one processor. In particular, the control unit can control the actuating device, in particular the actuating element or actuating elements, in order to control the position of the valve device or valve devices within the space of the housing.

Of particular advantage is the use of a processing device according to the invention or a processing system according to the invention in an incubator for liquid samples that can comprise biological particles. An incubator is a device used to create controlled conditions for various development and growth processes. Thus, temperature and/or humidity can be controlled by the means of the incubator.

In the figures, the subject-matter of the invention is schematically shown, wherein identical or similarly acting elements are usually provided with the same reference signs. Therein shows:

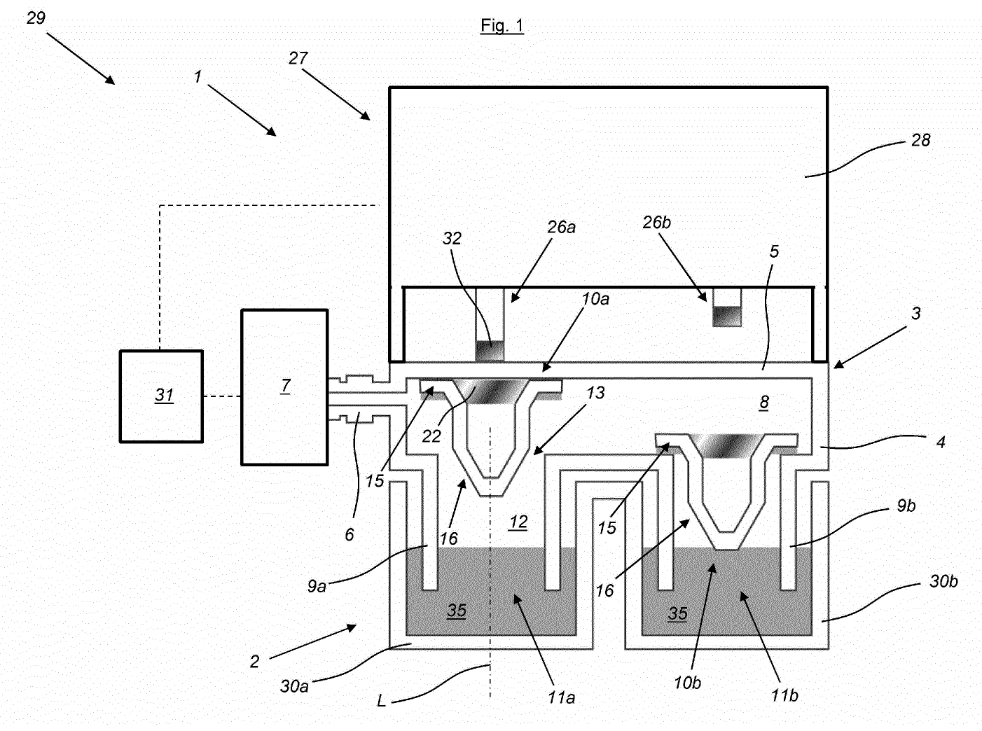

FIG. 1 a section view of a processing system with a processing device according to a first embodiment,

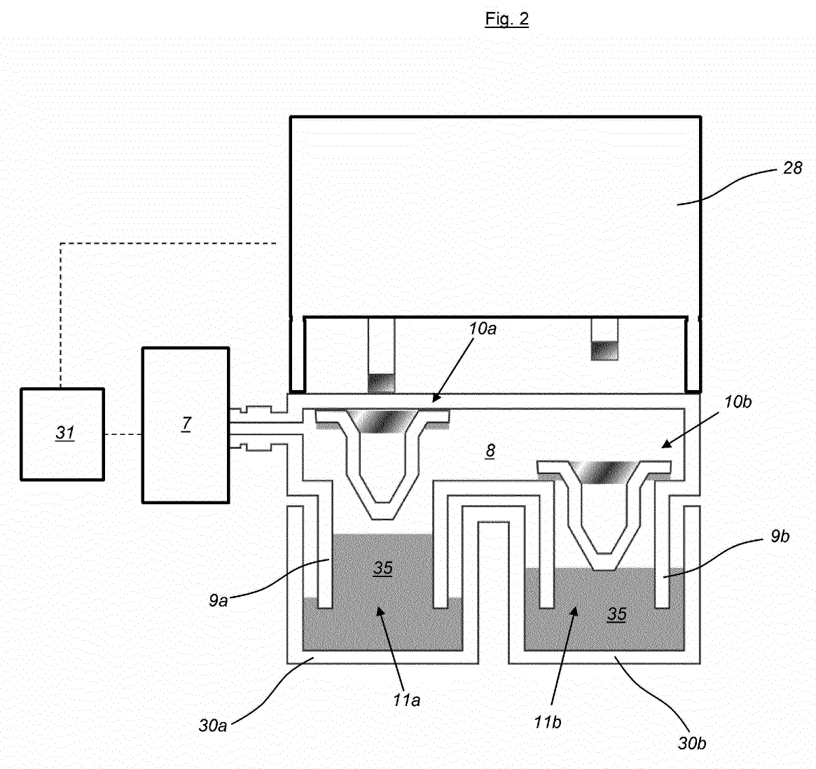

FIG. 2 a section view of processing system shown in FIG. 1 in a first processing state of the processing device,

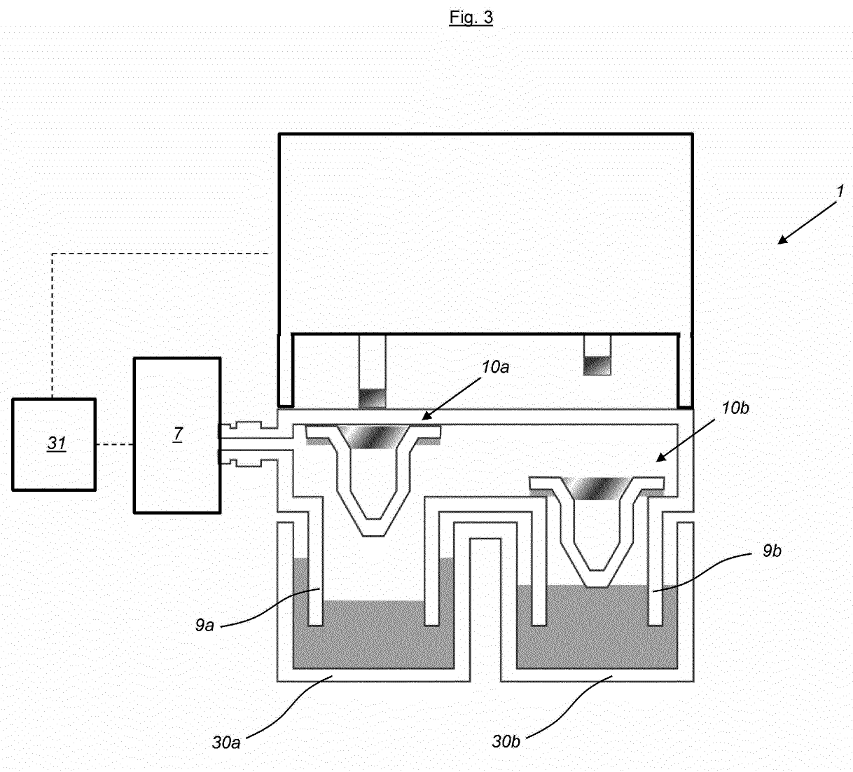

FIG. 3 a section view of processing system shown in FIG. 1 in a second processing state of the processing device,

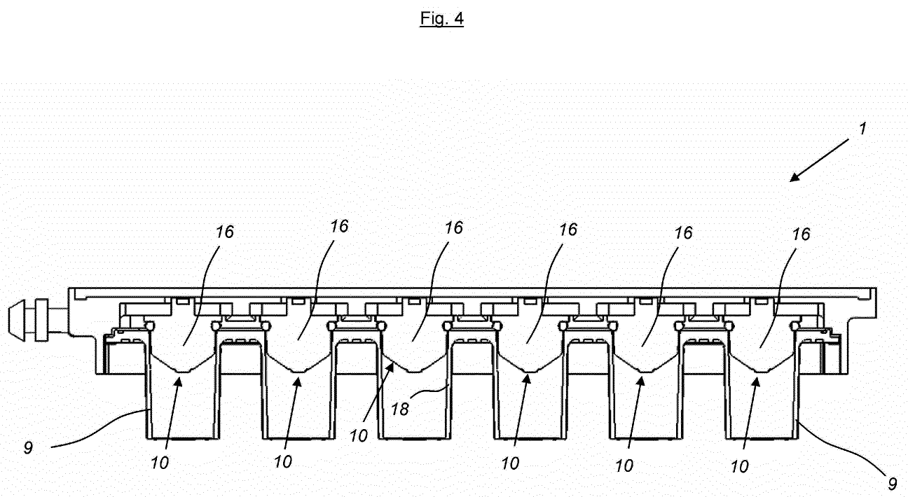

FIG. 4 a section view of a processing device according to a second embodiment,

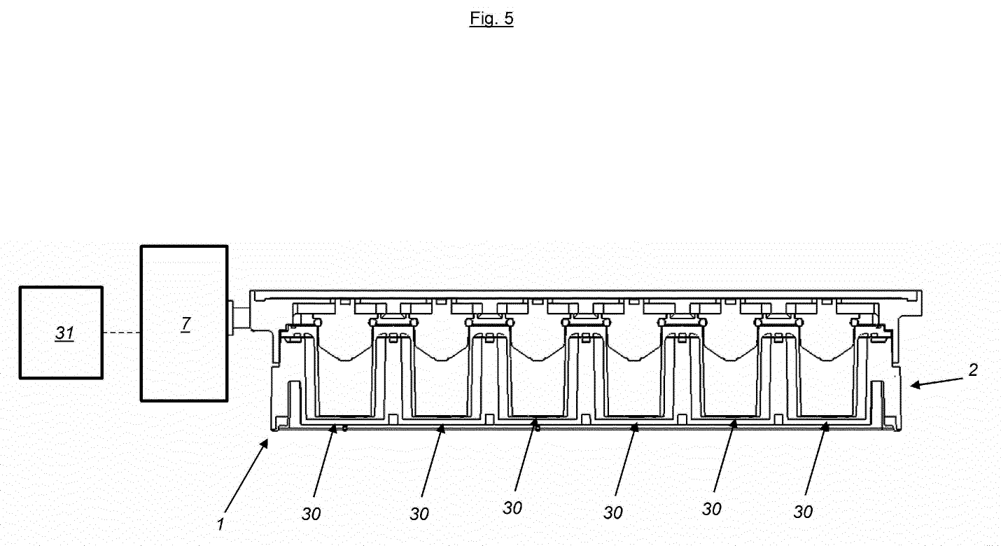

FIG. 5 a section view of a processing system with the processing device according to the second embodiment,

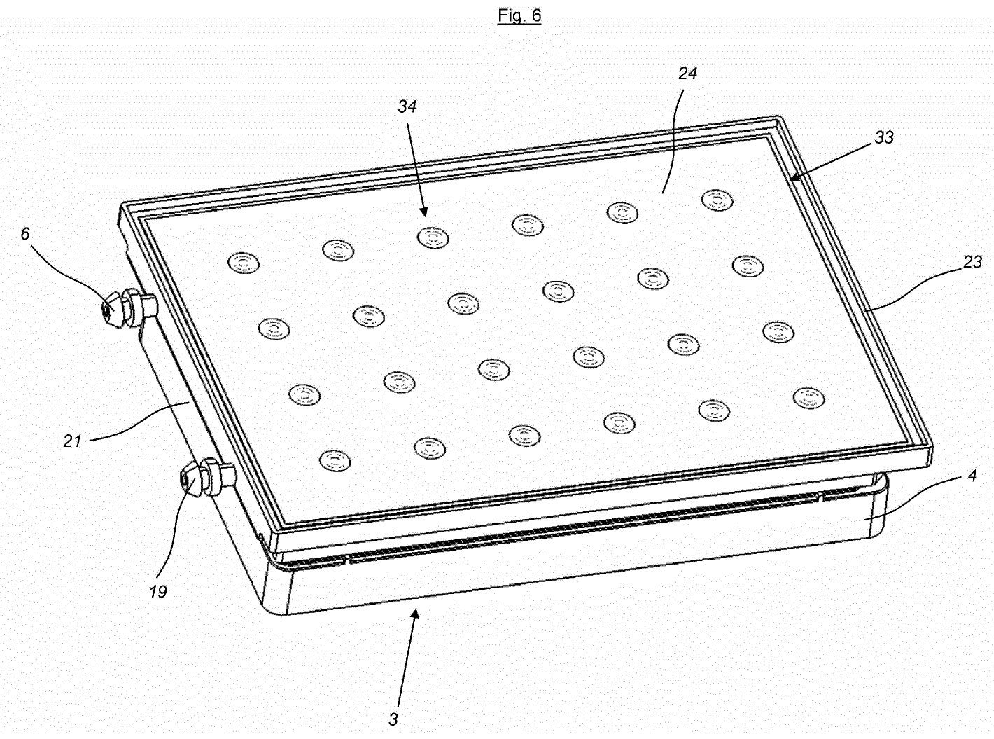

FIG. 6 a perspective view of the processing device according to the second embodiment without the actuating device,

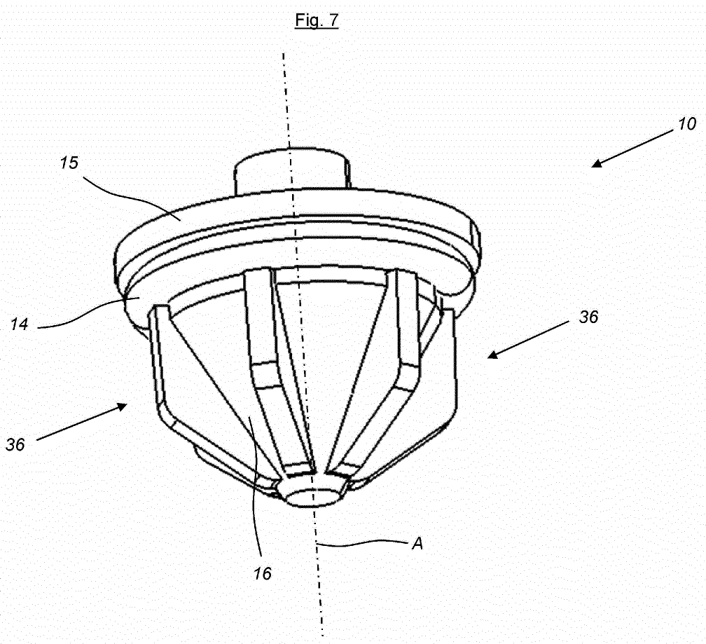

FIG. 7 a perspective view of the valve device used in the processing device according to the second embodiment,

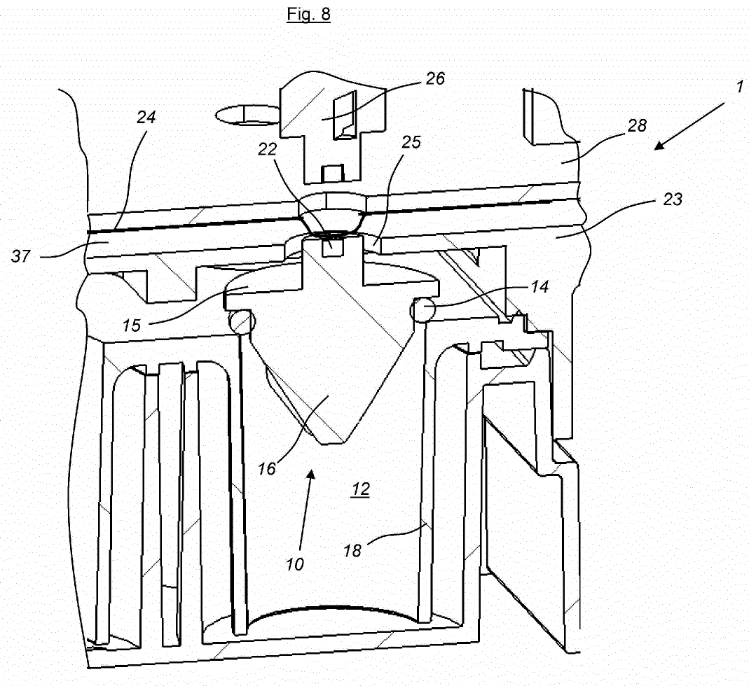

FIG. 8 a perspective view of a valve device being in a closed position of the processing device according to the second embodiment,

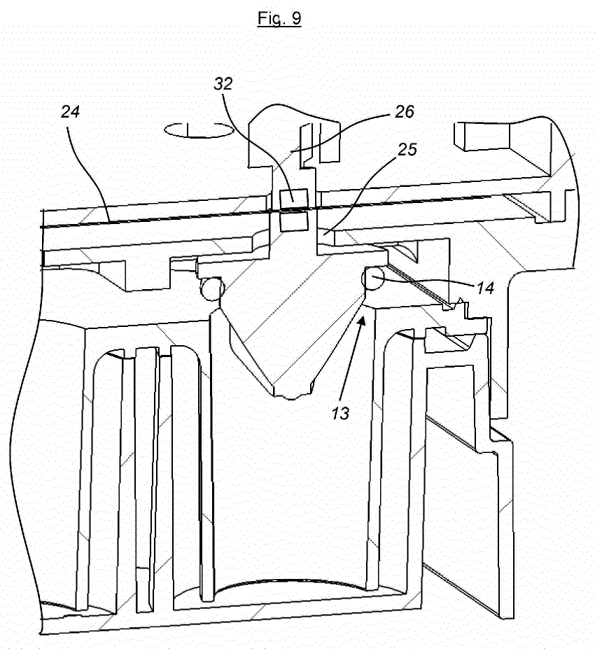

FIG. 9 a perspective view of a valve device being in an open state of the processing device according to the second embodiment,

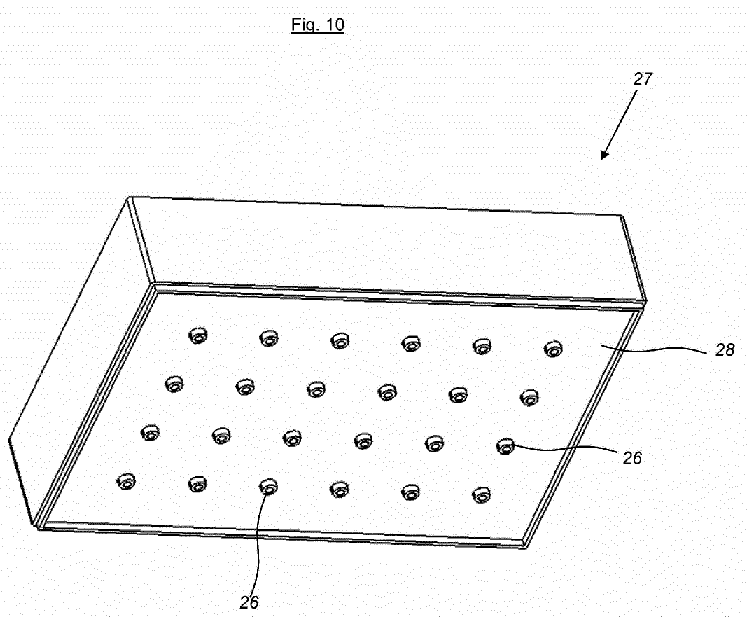

FIG. 10 a perspective of an actuating device and

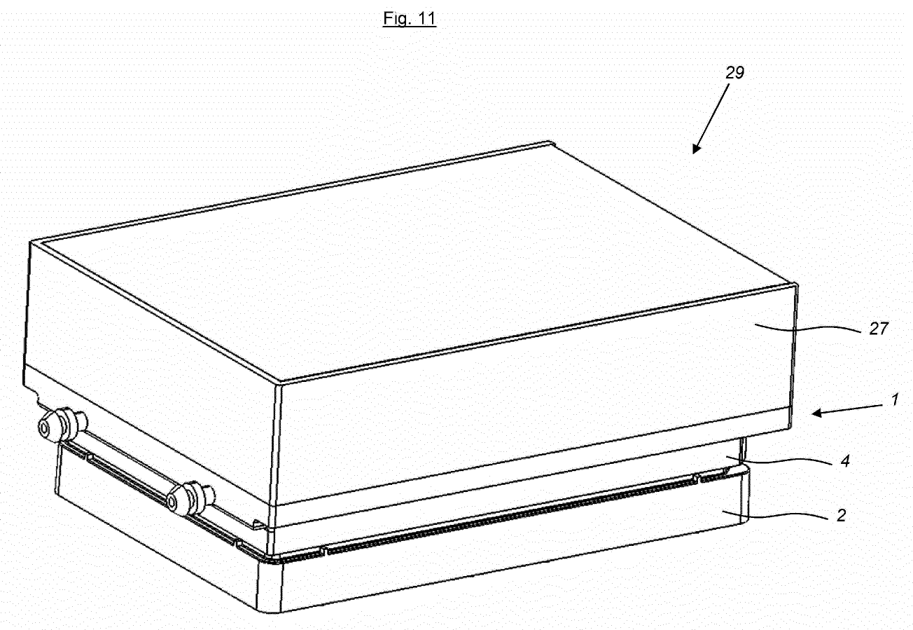

FIG. 11 a perspective view of a processing system according to the invention.

FIG. 1 shows a processing system 29 comprising a processing device 1 for processing of liquid sample contained in a multiwell carrier 2. Additionally, the processing system 29 comprises a processing unit 7 and a control unit 31.

The processing device 1 comprises a housing 3 with a bottom part 4 and a lid part 5. The bottom part 4 and the lid part 5 are fixed to each other. Additionally, the bottom part 4 and the lid part 5 delimit an inner space 8 of the housing 3. The processing device 1 also comprises a connection part 6 for fluidically connecting the processing device 1 with the processing unit 7. The connection part 6 is fluidically connected with the inner space 8. The housing 3 has several hollow extension tubes, namely a first extension tube 9a and a second extension tube 9b. Both extension tubes 9a, 9b extend from the bottom part 4 and are fluidically connected with the inner space 8.

Additionally, the processing device 1 comprises two valve devices, namely a first valve device 10a and a second valve device 10b. Both valve devices 10a, 10b are partly arranged in the inner space 8 of the housing 3. Additionally, both valve devices 10a, 10b are moveable between an open position in which the connection part 6 is fluidically connected with an outlet opening 11a, 11b of the extension tube 9a, 9b and a closed position in which the connection part 6 is fluidically disconnected from the outlet opening 11a, 11b of the extension tube 9a, 9b.

Each of the valve devices 10a, 10b is assigned to one extension tube 9a, 9b. In particular, the first valve device 10a is assigned to the first extension tube 9a and the second valve device 10b is assigned to the second extension tube 9b. Each of the extension tubes 9a, 9b is assigned to one well of the multiwell carrier 2. In particular, the first extension tube 9a extends into a first well 30a of the multiwell carrier 2 and the second extension tube extends into a second well 30b of the multiwell carrier 2. Both extension tubes 9a, 9b protrude into a liquid sample 35 arranged in the respective well 30a, 30b. That means, the outlet opening 11a, 11b of the respective extension tube 9a, 9b is arranged within the liquid sample 35. The extension tubes 9a, 9b are fixed to the bottom part 4 of the housing 3.

Both extension tubes 9a, 9b are designed in the same way so that in the following only one extension tube 9a, 9b is described. Likewise, both valve devices 10a, 10b are designed in the same way so that in the following only one valve device 10a, 10b is described.

In the embodiment shown in FIG. 1, the first valve device 10a is arranged in an open position and the second valve device 10b is arranged in a closed position. In the open position of the first valve device 10a the connection part 6 is fluidically connected with the first outlet opening 11a of the first extension tube 9a. In contrary to that, in the closed position of the second valve device 10b, the second outlet opening 11b of the second extension tube 9b is not fluidically connected with the connection part 6. The first valve device 10a moves from the closed position to the open position or vice versa along a length axis L of the first well 30a.

The first extension tube 9a comprises an inlet opening 13 that can be closed by the first valve device 10a. Whether the inlet opening 13 is closed or not depends on the position of the first valve device 10a. The inlet opening 13 and the outlet opening 11a are arranged at opposite ends of the first extension tube 9a. Both openings are part of a cavity 12 of the first extension tube 9a by means of which the inner space 8 is fluidically connected with the first well 30a, in particular the liquid sample contained in the first well 30a.

Both valve devices 10a, 10b are identically designed. Thus, in the following, the design of the first valve device 10a is described.

The first valve device 10a comprises a valve head 15 and a valve body 16. The valve body 16 comprises a cylindrical portion and a tapered portion. The tapered portion is arranged closer to the first outlet opening 11a than the cylindrical portion. The cylindrical portion is arranged between the tapered portion and the valve head 15. The valve head 15 and the valve body 16 are formed as a single part. The valve head 15 has a greater cross section than the cavity 12 in a plane that is perpendicular to a length axis L of the first extension tube 9a. This prevents the valve head 15 from entering the cavity 12. The valve device 10 comprises a magnetic element 22. The magnetic element 22 is a permanent magnet and is arranged at the valve head 15. In particular, the magnetic element 22 is fixed to the valve head 15. A part of the valve body 16 is arranged in the cavity 12 independent of whether the valve device 10 is arranged in the open or closed position. As is evident from FIG. 1 the valve body 16 has a cross section that is smaller than a cross section of the extension tube 9a, 9b delimiting the inner space 8. Thus, the valve body 16 is not in contact with the extension tube 9a, 9b so that the valve device 10a, 10b is not guided by the extension tube 9a, 9b, respectively.

The processing device 1 comprises an actuating device 27. The actuating device 27 is arranged on the housing 3. In particular, an outer rim of the actuating housing 28 is arranged on the housing 3. The actuating device 27 comprises an actuating housing 28 and two actuating elements, namely a first actuating element 26a and a second actuating element 26b. Both actuating elements 26 are moveable with respect to the actuating housing 28 and at least partially protrude from the actuating housing 28 towards the housing 3. Both actuating elements 26a, 26b have the same structure so that in the following only one actuating element is described.

The first actuating element 26a has a magnetic element 32 and is assigned to the first valve device 10a. The second actuating element 26b is assigned to the second valve device 10b. Thus, the number of actuating elements 26 correspond with the number of valve devices 10. The actuating elements 26a, 26b and the valve devices 10a, 10b are arranged opposite to each other with respect to the lid part 5.

In the embodiment shown in FIG. 1 the first actuating element 26a is arranged in an actuating position. This means, the first actuating element 26a is moved towards the first valve device 10a. In that case a magnet force resulting from the first actuating element 26a acts on the valve device 10a leading to the movement of the first valve device 10a to the open position. The second actuating element 26b is arranged in a non-actuating position. In said position no force acts on the second valve device 10b or the force acting on the second valve device 10b is not sufficient to move the second valve device 10b from the closed position to the open position. Thus, the second valve device 10b remains in the closed position.

FIG. 2 shows a section view of processing system shown in FIG. 1 in a first processing state of the processing device 1. The control unit 31 causes the processing unit 7 to apply a negative pressure in the inner space 8. As the first valve device 10a is arranged in an open position, the negative pressure also acts on the first outlet opening 11a of the first extension tube 9a, resulting in the suction of liquid sample 35 into the first extension tube 9a. As the second valve device 10b is arranged in the closed position, the negative pressure of the inner space 8 does not apply to the second outlet opening 11b of the second extension tube 9b so that the liquid sample level of the liquid sample 35 arranged in the second well 30b does not change in comparison the situation shown in FIG. 1. The liquid sample 35 arranged in the second well 30b is not processed.

FIG. 3 shows a section view of the processing system 29 shown in FIG. 1 in a second processing state of the processing device 1. In the second processing state, the control unit 31 acts on the processing unit 7 to apply a positive pressure in the inner space 8. As the first valve device 10a remains in the open state, the positive pressure acts on the first outlet opening 11a and thus on the liquid sample arranged in the first extension tube 9a leading to the discharge of liquid sample from the first extension tube 9a into the first well 30a. As the second valve device 10b remains in the closed position the positive pressure does not act on the liquid sample arranged in the second well 10b so that the liquid sample level remains the same as in the first processing state shown in FIG. 2.

The embodiment shown in FIG. 1-3 has only two extension tubes 9a, 9b. However, it is clear that the embodiment can have more than two extension tubes 9a, 9b.

FIG. 4 shows a section view of a part processing device 1 according to a second embodiment. In particular, the actuating device 27 is not shown in FIG. 4. The processing device 1 according to the second embodiment differs from the processing device 1 according to the first embodiment in the valve device 10 and in the structure of the lid part. A further difference is that the processing device 1 according to the second embodiment comprises more valve devices 10 and extension tubes 9 than the processing device 1 according to the first embodiment. The valve device 10 is in contact with a tube wall 18 so that the tube wall 18 guides the movement of the valve device 10. The valve device 10 used in the processing device 1 is shown in more detail in FIG. 7.

FIG. 5 shows a section view of a processing system with the processing device 1 according to the second embodiment shown in FIG. 4. As is evident from FIG. 5 the multiwell carrier 2 comprises more wells 30 than the multiwell carrier 2 shown in FIGS. 1 to 3. In particular, in the shown embodiment the number of wells 30 corresponds with the number of extension tubes 9.

FIG. 6 shows a perspective view of the processing device 1 according to the second embodiment without the actuating device 27. The processing device 1 comprises a further connection part 19. Both the connection part 6 and the further connection part 19 extend from the same housing side 21. Alternatively, in a non-shown embodiment, the connection part 6 and the further connection part 19 can extend from different housing sides.

The lid part comprises a lid frame 23 and a membrane 24 that is arranged on the lid frame 23. The lid frame 23 is fixed to the bottom part 4. The membrane 24 is elastic and the outer rim of the membrane 24 is fixed to the lid frame 23 along the circumference of the membrane 24. In the second embodiment the valve device 10 is fixed to the membrane 24, which is explained below more in detail. However, due to the connection of the valve device 10 to the membrane 24 and the elasticity of the membrane 24 depressions 34 are evident in FIG. 6 at positions at which the valve device 10 is connected to the membrane 24.

FIG. 7 shows a perspective view of the valve device 10 used in the processing device 1 according to the second embodiment. The valve device 10 comprises a tapered valve body 16. The sealing element 14 is arranged at one end of the valve body 16.

Additionally, the valve device 10 comprises several guiding portions 36 that are arranged at the valve body 16. The guiding portions 36 are arranged at the valve body 16 in a distance to each other along a circumferential direction of the valve body 16. The guiding portions 36 extend along length axis A of the valve device 10. The guiding portions 36 comprise a part extending along the length axis A that has a constant radius with respect to the length axis A, respectively. Said part is in contact with a tube wall 18 as shown in FIG. 4. That means, said part is used for guiding the valve device 10. Additionally, the valve device 10 comprises another part that extends along the length axis A and has a decreasing radius along the length axis A.

FIG. 8 shows an enlarged perspective view of a valve device 10 being in a closed position of the processing device 1 according to the second embodiment. A difference between the processing device 1 according to the second embodiment and the processing device 1 according to the first embodiment is the structure of the lid part as described above for FIG. 6 and the structure of the valve device 10 as discussed below.

The valve device 10 is arranged in a closed position and comprises a sealing element 14. The sealing element 14 is arranged at the valve head 15 and surrounds the inlet opening 13. The lid frame 23 comprises a hole 25 and a part of the valve head 15 is arranged in said hole 25. The hole 25 is covered by the membrane 24. The top part of the valve head 15 is fixed to the membrane 24. An air gap 37 is provided between the lid frame 23 and the membrane 24. Alternatively, in a non-shown embodiment, the membrane 24 can be directly arranged on the lid frame 23 so that there is no air gap between the lid frame 23 and the membrane 24.

The actuating device 27 is arranged on the processing device 1. In particular, the actuating housing 28 is arranged on the processing device 1. The actuating element 26 is arranged in a non actuating position in which it does not exert a force on the valve device 10 such that the valve device 10 moves to the open position. The actuating element 26 can move to an actuating position as shown in FIG. 9.

FIG. 9 shows an enlarged perspective view of a valve device 10 being in an open state of the processing device 1 according to the second embodiment. The actuating element 26 was moved to the actuating position. In said position, the actuating element 26 is in contact with the membrane 24. The magnetic element 32 of the actuating element 26 acts on the valve device 10 such that the valve device 10 is moved to the open position. In said position the sealing element 14 is not in contact anymore with the bottom part 4 and/or the extension tube 9 so that the inlet opening 13 is not closed anymore by the valve device 10. The actuating device 27 can be identical to the actuating device 27 described above in connection with the processing device 1 according to the first embodiment.

FIG. 10 shows a perspective view of an actuating device 27 from below. It is evident that the actuating device 27 comprises a plurality of actuating elements 26 protruding from the actuating housing 28. The number of actuating elements 26 corresponds with the number of valve devices 10 arranged in the inner space 8 of the housing 3. Additionally, the actuating elements 26 are arranged such that they can exert force on the valve device 10 to which they are assigned. The actuating device 27 can be used in processing devices 1 with valve devices 10 as described above for processing devices 1 according to the first or the second embodiment. The actuating elements 26 are arranged in a matrix structure.

FIG. 11 a perspective view of a processing system 29. In FIG. 10 the processing unit 7 and the control unit 31 are not shown. The processing device 1 is arranged on the multiwell carrier 2. In particular, the processing device 1 is arranged directly on the multiwell carrier 2. The actuating device 27 is arranged on the housing 3. The processing device 1 can have valve devices 10 as described above for the processing device 1 according to the first or the second embodiment.

### REFERENCE SIGNS

1 processing device
2 multiwell carrier
3 housing
4 bottom part
5 lid part
6 connection part
7 processing unit
8 inner space
9 extension tube
9a first extension tube
9b second extension tube
10 valve device
10a first valve device
10b second valve device
11a first outlet opening
11b second outlet opening
12 cavity
13 inlet opening
14 sealing element
15 valve head
16 valve body
18 tube wall
19 further connection part
21 housing side
22 magnetic element of valve device
23 lid frame
24 membrane
25 hole
26 actuating element
26a first actuating element
26b second actuating element
27 actuating device
28 actuating housing
29 processing system
30 well
30a first well
30b second well
31 control unit
32 magnetic element of actuating element
34 depression
35 liquid sample
36 guiding portion
37 air gap
A Length axis of valve device
L Length axis

1.-24. (canceled)

25. A processing device for processing of a liquid sample contained in a multiwell carrier, wherein the processing device comprises:
a housing with a bottom part and a lid part, wherein the bottom part and the lid part delimit an inner space of the housing; and
a connection part for fluidically connecting the processing device with a processing unit wherein the connection part is fluidically connected with the inner space;
wherein the housing has several hollow extension tubes, which extend from the bottom part and which are fluidically connected with the inner space; and
wherein the processing device comprises at least one valve device that is at least partly arranged in the inner space of the housing and is moveable between an open position in which the connection part is fluidically connected with an outlet opening of an extension tube and a closed position in which the valve device fluidically disconnects the connection part from the outlet opening of the extension tube.

26. The processing device according to claim 25, wherein:
a. the valve device is partially arranged in a cavity of the extension tube; and/or
b. the valve device closes an inlet opening of the extension tube when the valve device is arranged in the closed position; and/or
c. the valve device moves translatory from the open position to the closed position or vice versa; and/or
d. the valve device moves from the open position to the closed position or vice versa along a length axis of the extension tube; and/or
e. the valve device moves from the open position to the closed position by means of force of gravity.

27. The processing device according to claim 25, wherein:
a. the valve device has a sealing element for sealing an inlet opening of the extension tube when the valve device is arranged in the closed position; and/or
b. the valve device has a sealing element that surrounds an inlet opening of the extension tube when the valve device is arranged in the closed position.

28. The processing device according to claim 25, wherein the valve device has a valve head and a valve body, wherein:
a. the valve head is configured such that the valve head cannot enter a cavity of the extension tube; and/or
b. at least one guiding portion is arranged at a valve body wherein the guiding portion is guided by a tube wall of the extension tube; and/or
C. several guiding portions are arranged at a valve body wherein the guiding portions are arranged at a distance to each other in a circumferential direction of the valve body.

29. The processing device according to claim 28, wherein:
a. the valve body has a cylindrical portion and/or a tapered portion; and/or
b. an end of the valve body distant to the valve head has a tapered portion.

30. The processing device according to claim 25, wherein the processing device comprises a further connection part for connecting the inner space with a measurement device.

31. The processing device according to claim 30, wherein:
a. the connection part and/or the further connection part protrude from the lid part and/or bottom part; and/or
b. the connection part and/or the further connection part protrude from the same housing side.

32. The processing device according to claim 25, wherein:
a. the valve device has a magnetic element; and/or
b. the valve device has a magnetic element that it is arranged at an end of the valve device distant to the extension tube.

33. The processing device according to claim 25, wherein the lid part comprises a lid frame and a membrane arranged on at least a part of the lid frame.

34. The processing device according to claim 33, wherein:
a. the lid frame is fixed to the bottom part; and/or
b. the membrane is at least partially connected to the lid frame; and/or
c. the lid frame comprises a hole wherein a part of the valve device extends through the hole and is connected with the membrane; and/or
d. the lid frame comprises a hole, wherein a part of the membrane enters the hole when the valve device is arranged in the closed position; and/or
e. the lid frame is stiffer than the membrane.

35. The processing device according to claim 33, wherein:
a. the valve device is fixed to the lid part; and/or
b. the valve device is fixed to the membrane.

36. The processing device according to claim 25, wherein the valve device is movable relative to the lid part and/or the bottom part.

37. The processing device according to claim 36, wherein the valve device is in contact with the lid part when the valve device is arranged in the open position and is not in contact with the lid part when the valve device is arranged in the closed position.

38. The processing device according to claim 25, wherein the processing device comprises an actuating element that is configured to act on the valve device so that the valve device moves from the closed position to the open position.

39. The processing device according to claim 38, wherein:
a. the actuating element is moveable relative to the processing device; or
b. the actuating element is arranged in the same position independent of whether the valve device is in the open position or the closed position.

40. The processing device according to claim 38, wherein:
a. the actuating element and the valve device are arranged opposite to each other with respect to the lid part; and/or
b. the actuating element comprises a magnetic element.

41. The processing device according to claim 38, wherein the processing device comprises an actuating device, wherein:
a. the actuating device is arranged on the lid part; and/or
b. the actuating device comprises an actuating housing wherein the actuating element is arranged at least partly in an inner space of the actuating housing.

42. The processing device according to claim 25, wherein:
a. the processing device comprises several valve devices wherein each of the valve devices is assigned to an extension tube; and/or
b. the valve devices are moveable independently of each other; and/or
c. the processing device comprises several actuating elements wherein each of the actuating elements is assigned to a valve device.

43. A processing system comprising a processing device according to claim 25 and a multiwell carrier, wherein the processing device is arranged such that at least one extension tube extends in a well containing the liquid sample of the multiwell carrier.

44. The processing system according to claim 43, wherein:
a. the processing system comprises a processing unit wherein the processing unit is configured to apply a positive pressure or a negative pressure in the inner space of the processing device; and/or
b. the processing device is arranged on the multiwell carrier.
